(19) United States
(12) Patent Application Publication
(10) Pub. No.: US 2009/0209426 A1
(43) Pub. Date: Aug. 20, 2009

(54) HERBICIDAL MIXTURE

(76) Inventors: Jose Evanil Da Silva, Vinhedo (BR); Edison Hidalgo, Newark, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/294,094

(22) PCT Filed: May 8, 2007
(86) PCT No.: PCT/US07/11066

§ 371 Date: Sep. 23, 2008

Related U.S. Application Data
(60) Provisional application No. 60/800,252, filed on May 12, 2006.

(51) Int. Cl. A01N 47/28; C07C 317/44; A01P 13/00

(52) U.S. Cl. 504/148; 568/30

(57) ABSTRACT

A mixture of diuron with mesotrione optionally containing other herbicides such hexazinone is disclosed as useful for controlling undesired vegetation. Also disclosed is a herbicidal composition comprising diuron, mesotrione and optionally other herbicides such as hexazinone and at least one additional component selected from the group consisting of surfactants, solid diluents and liquid diluents. Further disclosed is a method of controlling undesired vegetation comprising applying to the locus of the vegetation a herbicidally effective amount of the mixture.

### FIELD OF THE INVENTION

This invention relates to mixtures of herbicidal compounds and their compositions, and methods using the mixtures and compositions for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, corn (maize), potato, wheat, barley, tomato and plantation crops such as sugarcane, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new products that are more effective, less costly, less toxic, environmentally safer or have different modes of action.

Combinations of herbicides are typically used to broaden the spectrum of plant control or enhance the level of control of any given species through additive effect. Furthermore, certain rare combinations unexpectedly give a greater-than-additive or synergistic effect on weeds or a less-then-additive or safening effect on crops.

Diuron (N′-(3,4-dichlorophenyl)-N,N-dimethylurea), disclosed in U.S. Pat. No. 2,655,445, has become well-known as a herbicide providing control of undesired vegetation (i.e. weeds) in many crops, including asparagus, pineapple, banana, sugarcane, cotton, cranberry, peppermint, alfalfa and other forage legumes, cereals, maize (corn), sorghum, perennial grass-seed crops, olives, citrus and other fruit trees, and grapes and other vines. Diuron is also used as an effective herbicide to control weeds in areas other than crops, such as on industrial sites, on railroad and other transportation rights-of-way, around farm buildings, and on the banks of irrigation and drainage ditches. Although diuron is often applied as a directed application to avoid contact with the foliage of crops, because of diuron's greater activity against germinating rather than established plants, diuron can be satisfactorily applied under some circumstances over the top on established alfalfa, birdsfoot trefoil, asparagus, grass seed crops, oats, red clover, sugarcane, wheat and pineapple. However, because diuron's herbicidal effect is strongest against germinating plants, obtaining satisfactory control of established weeds with postemergence applications of diuron is more difficult to achieve than preemergence control of germinating weeds.

Mesotrione (2-[4-(methylsulfonyl)-2-nitrobenzoyl]-1,3-cyclohexanedione), disclosed in U.S. Pat. No. 5,006,158, is a relatively new herbicide developed for selective control of broad-leaved weeds along with some grass weeds in maize. Mesotrione is also now being used in cranberry crops for control of broad-leaved weeds. Mesotrione is effective for both preemergence and postemergence application, and unlike diuron, lower application rates are typically needed for satisfactory control from postemergence compared to preemergence applications. Although mesotrione is useful for postemergent weed control, it is weak on some weeds, particularly grasses. For control of these weeds, new solutions are desirable.

### SUMMARY OF THE INVENTION

This invention provides a mixture comprising diuron (the compound of Formula I)

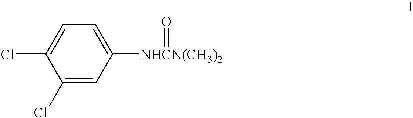

and at least one compound selected from mesotrione (the compound Formula II)

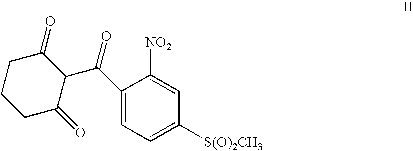

and salts thereof.

This invention also provides a herbicidal composition comprising diuron, at least one compound selected from mesotrione and salts thereof, and at least one additional component selected from the group consisting of surfactants, solid diluents and liquid diluents. This invention also provides a method of controlling undesired vegetation comprising applying to the locus of the vegetation a herbicidally effective amount of the aforesaid mixture (e.g., as a herbicidal composition thereof). This invention also relates to the aforesaid mixture and composition further comprising a biologically effective amount of at least one additional biologically active compound or agent, and method of controlling undesired vegetation comprising applying to the locus of the vegetation a herbicidally effective amount of said mixture or composition.

### DETAILS OF THE INVENTION

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

Embodiments of the present invention as described in the Summary of the Invention include (where mesotrione as used in the following Embodiments means at least one compound selected from mesotrione and salts thereof, unless specified otherwise):
Embodiment 1. A mixture or composition described in the Summary of the Invention comprising diuron and mesotrione and further comprising hexazinone.
Embodiment 2. A composition described in the Summary of the Invention which is a homogeneous mixture comprising two groups of substantially cylindrical granules, the groups having different herbicide contents, the first group of granules comprising diuron and at least one solid diluent, and the second group of granules comprising a least one compound selected from mesotrione and salts thereof and at least one solid diluent, the granules within each group having substantially uniform diameters and longitudinal lengths of from 1 to 8 times the diameter with the average length of the granules being from 1.5 to 4 times the diameter, and the average diameter of the first and second group differing from the first group by no more than 30%.
Embodiment 3. The composition of Embodiment 2 wherein the homogeneous mixture further comprises a third group of substantially cylindrical granules having a biologically active compound or agent content differing from the contents of the first and second groups, the third group of granules comprising an additional biologically active compound or agent and at least one solid diluent, the granules within the third group having substantially uniform diameters and longitudinal lengths of from 1 to 8 times the diameter with the average length of the granules being from 1.5 to 4 times the diameter, and the average diameter of the third group differing from the first and second groups by no more than 30%.
Embodiment 4. The composition of Embodiment 3 wherein said additional biologically active compound or agent comprises hexazinone.
Embodiment 5. A method described in the Summary of the Invention wherein the mixture or composition applied to the locus of the undesired vegetation comprises hexazinone in addition to diuron and mesotrione.
Embodiment 6. A method described in the Summary of the Invention wherein the undesirable vegetation is a monocot plant species.
Embodiment 7. The method of Embodiment 6 wherein the undesirable vegetation is a plant species in the family Poaceae.
Embodiment 8. The method of Embodiment 7 wherein the undesirable vegetation is a plant species in the genus Digitaria.
Embodiment 9. The method of Embodiment 8 wherein the undesirable vegetation is Digitaria nuda or Digitaria horizontalis.
Embodiment 10. The method of Embodiment 9 wherein the undesirable vegetation is Digitaria nuda.
Embodiment 11. The method of Embodiment 6 wherein the undesirable vegetation is a plant species in the family Cyperaceae.
Embodiment 12. The method of Embodiment 11 wherein the undesirable vegetation is a plant species in the genus Cyperus.
Embodiment 13. The method of Embodiment 12 wherein the undesirable vegetation is Cyperus esculentus.
Embodiment 14. The method of Embodiment 12 wherein the undesirable vegetation is Cyperus rotundus.
Embodiment 15. A method described in the Summary of the Invention wherein the herbicidal mixture or composition comprising diuron and mesotrione is applied after emergence of the undesired vegetation.
Embodiment 16. A method described in the Summary of the Invention wherein the locus of the undesired vegetation is a citrus crop.
Embodiment 17. A method described in the Summary of the Invention wherein the locus of the undesired vegetation is a coffee crop.
Embodiment 18. A method described in the Summary of the Invention wherein the locus of the undesired vegetation is a maize crop.
Embodiment 19. A method described in the Summary of the Invention wherein the locus of the undesired vegetation is a sugarcane crop.
Embodiment 20. A method described in the Summary of the Invention wherein the locus of the undesired vegetation is a cranberry crop.
Embodiment 21. A method described in the Summary of the Invention wherein the locus of the undesired vegetation is other than a crop.

Embodiments of this invention can be combined in any manner.

Of note is a method, mixture or composition as described in the Summary of the Invention or in any one of Embodiments 1-21 wherein the mesotrione is in the free-acid form (i.e. not as a salt).

In the subsequent disclosure, recitation of “mesotrione” refers to mesotrione in one or more of its forms (i.e. free acid, salts) unless precluded by the context or specified otherwise.

Diuron (Formula I) is commercially available in herbicidal compositions sold by a variety of companies including DuPont (e.g., KARMEX® Herbicide). Although diuron is most conveniently obtained as a commercial product, it can be prepared by methods described in U.S. Pat. No. 2,655,445.

Mesotrione (Formula II) is commercially available in herbicidal compositions sold by Syngenta (e.g., CALLISTOO® Herbicide). Although mesotrione is most conveniently obtained as a commercial product, it can be prepared as described in U.S. Pat. No. 5,006,158.

As mesotrione is acidic (pKₐ 3.12), the mixture of the present invention can include mesotrione as one or more salts. The salts of mesotrione include those formed with organic bases (e.g., pyridine, ammonia, or triethylamine) or inorganic bases (e.g., hydrides, hydroxides, or carbonates of sodium, potassium, lithium, calcium, magnesium or barium). Besides containing metal cations such as alkali metal (e.g., sodium, potassium, lithium) and alkaline earth metal (e.g., calcium, magnesium, barium), salts of mesotrione can also include sulfonium, sulfoxonium or quaternary ammonium cations. Salts of mesotrione can be prepared in a number of ways known in the art for preparing salts of acidic organic compounds. For example, metal salts can be made by contacting mesotrione in its acid form (Formula II) with a solution of an alkali or alkaline earth metal salt having a sufficiently basic anion (e.g., hydroxide, alkoxide, carbonate or hydride). Quaternary ammonium salts can be made by similar techniques.

Salts of mesotrione can also be prepared by exchange of one cation for another. Cationic exchange can be effected by direct contact on an aqueous solution of a salt of mesotrione (e.g., alkali or quaternary ammonium salt) with a solution containing the cation to be exchanged. The method is most effective when the desired salt containing the exchanged cation is insoluble in water and can be separated by filtration.

Exchange may also be effected by passing an aqueous solution of a salt of a compound of mesotrione (e.g., an alkali metal or quaternary ammonium salt) through a column packed with a cation-exchange resin containing the cation to be exchanged for that of the original salt, and the desired product is eluted from the column. This method is particularly useful when the desired salt is water soluble (e.g., potassium, sodium or calcium salt).

### Formulation/Utility

The mixture of this invention can be formulated in a number of ways:
(a) diuron (Formula I) and mesotrione (Formula II, including salts thereof) can be formulated into separate herbicidal compositions and applied separately or applied simultaneously (e.g., as a tank mix) in an appropriate weight ratio; or
(b) diuron (Formula I) and mesotrione (Formula II, including salts thereof) can be formulated together in the desired weight ratio in a single herbicidal composition.
   Additional biologically active compounds or agents included in the mixture can similarly be formulated and applied separately or together.

Diuron, mesotrione and their mixtures, including their mixtures with other biologically active compounds or agents such as hexazinone will generally be used in a formulation (i.e. herbicidal composition) with an agriculturally suitable carrier comprising at least one of a liquid diluent, a solid diluent or a surfactant. The formulation or composition ingredients are selected to be consistent with the physical properties of the active ingredients, mode of application and environmental factors such as soil type, moisture and temperature. Useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films (including seed coatings), and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations will typically contain effective amounts of active ingredients, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders. | | | |
| Suspensions, Emulsions, | 1-50 | 40-99 | 0-50 |
| Solutions (including | | | |
| Emulsifiable Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Typical solid diluents are described in Watkins et al., Handbook of Insecticide Dust Diluents and Carriers, 2nd Ed., Dorland Books, Caldwell, N.J. Typical liquid diluents are described in Marsden, Solvents Guide, 2nd Ed., Interscience, New York, 1950. McCutcheon's Detergents and Emulsifiers Annual, Allured Publ. Corp., Ridgewood, New Jersey, as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, polyethoxylated alcohols, polyethoxylated alkylphenols, polyethoxylated sorbitan fatty acid esters, dialkyl sulfosuccinates, alkyl sulfates, alkylbenzene sulfonates, organosilicones, N,N-dialkyltaurates, lignin sulfonates, naphthalene sulfonate formaldehyde condensates, polycarboxylates, glycerol esters, polyoxyethylene/polyoxypropylene block copolymers, and alkylpolyglycosides where the number of glucose units, referred to as degree of polymerization (D.P.), can range from 1 to 3 and the alkyl units can range from C₆ to C₁₄ (see Pure and Applied Chemistry 72, 1255-1264). Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, propylene carbonate, dibasic esters, paraffins, alkylbenzenes, alkylnaphthalenes, glycerine, triacetine, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, acetates such as hexyl acetate, heptyl acetate and octyl acetate, and alcohols such as methanol, cyclohexanol, decanol, benzyl and tetrahydrofurfuryl alcohol.

Useful formulations of this invention may also contain materials well known to those skilled in the art as formulation aids such as antifoams, film formers and dyes. Antifoams can include water dispersible liquids comprising polyorganosiloxanes like Rhodorsil® 416. The film formers can include polyvinyl acetates, polyvinyl acetate copolymers, polyvinylpyrrolidone-vinyl acetate copolymer, polyvinyl alcohols, polyvinyl alcohol copolymers and waxes. Dyes can include water dispersible liquid colorant compositions like Pro-lzed® Colorant Red. One skilled in the art will appreciate that this is a non-exhaustive list of formulation aids. Suitable examples of formulation aids include those listed herein and those listed in McCutcheon's 2001, Volume 2: Functional Materials published by MC Publishing Company and PCT Publication WO 03/024222.

Solutions, including emulsifiable concentrates, can be prepared by simply mixing the ingredients. Dusts and powders can be prepared by blending and, usually, grinding as in a hammer mill or fluid-energy mill. Suspensions are usually prepared by wet-milling; see, for example, U.S. Pat. No. 3,060,084. Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

Homogeneous mixtures (i.e. blends) of substantially cylindrical granules (also described as pellets), which can be formed by extrusion or pelletization, as taught in U.S. Pat. No. 6,022,552 provide a particularly convenient means of preparing the herbicidal composition of the present invention, as this technology allows easily varying the ratio of diuron to mesotrione and also other active ingredients such as hexazinone after extrusion and sizing or pelletization. U.S. Pat. No. 6,270,025 teaches a commercially useful method for sizing extruded granules in length ranges suitable for these homogeneous mixtures. The homogeneous mixture described in U.S. Pat. No. 6,022,552 comprises two or more groups of solid pesticide (e.g., herbicide) granules, wherein one group has one pesticide or pesticide content and one or more other groups have a different pesticide, a different pesticide content or an inert content, the granules within each group being formed by extrusion or pelletization and being substantially cylindrical in shape, having substantially uniform diameters and longitudinal lengths 1 to 8 times the diameter with the average length of the granules being 1.5 to 4 times the diameter, and the average diameter of each group differing from another group by no more than 30%. What is meant by substantially cylindrical is rod like or tubular wherein the cross-sectional shape may be circular, octagonal, rectangular, or any other conceivable shape and wherein the longitudinal surface is spiral, curved, or straight. The difference in average diameter is calculated by subtracting the average diameter of the granules in the group having the smaller diameter from the average diameter of the granules in the group having the larger diameter, then dividing the calculated difference by the average diameter of the granules in the group having the smaller diameter, and finally multiplying the calculated quotient by 100%. The inert content relates to ingredients other than the pesticidal active ingredients. In homogeneous mixtures of substantially cylindrical granules, granules comprising an active ingredient typically also comprise at least one solid diluent, and most typically also comprise at least one surfactant.

For the compositions of the present invention, in the homogeneous mixture one group of granules can comprise diuron and a second group of granules can comprise mesotrione. The homogeneous mixture can also comprise one or more additional groups of granules with other pesticide or inert content. For example, the first group of granules can comprise diuron, the second group of granules can comprise mesotrione, and a third group of granules can comprise another biologically active compound or agent such as hexazinone. Alternatively, two or more biologically active compounds or agents can be contained in one of the groups of granules. For example, the first group of granules can comprise diuron and hexazinone, and the second group of granules can comprise mesotrione. Alternatively, the first group of granules can comprise diuron and mesotrione, and the second group of granules can comprise hexazinone. As another example, the first group of granules can comprise hexazinone and mesotrione, and the second group of granules can comprise diuron.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways.

### Example A

High Strength Concentrate

| | |
|---|---|
| diuron | 98.3% |
| mesotrione | 0.2% |
| silica aerogel | 0.5% |
| synthetic amorphous fine silica | 1.0%. |

### Example B

High Strength Concentrate

| | |
|---|---|
| diuron | 65.7% |
| mesotrione | 32.8% |
| silica aerogel | 0.5% |
| synthetic amorphous fine silica | 1.0%. |

### Example C

Wettable Powder

| | |
|---|---|
| diuron | 62.9% |
| mesotrione | 2.1% |
| dodecylphenol polyethylene glycol ether | 2.0% |
| sodium ligninsulfonate | 4.0% |
| sodium silicoaluminate | 6.0% |
| montmorillonite (calcined) | 23.0%. |

### Example D

Granule

| | |
|---|---|
| diuron | 8.3% |
| mesotrione | 1.7% |
| attapulgite granules (low volatile matter, | 90.0%. |
| 0.71/0.30 mm; U.S.S. No. 25-50 sieves) | |

### Example E

Aqueous Suspension

| | |
|---|---|
| diuron | 24.0% |
| mesotrione | 1.0% |
| hydrated attapulgite | 3.0% |
| crude calcium ligninsulfonate | 10.0% |
| sodium dihydrogen phosphate | 0.5% |
| water | 61.5%. |

### Example F

Extruded Granule (Pellet)

| | |
|---|---|
| diuron | 24.1% |
| mesotrione | 0.9% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0%. |

### Example G

Extruded Granule (Pellet)

| | |
|---|---|
| diuron | 18.7% |
| mesotrione | 1.0% |
| hexazinone | 5.3% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0%. |

### Example H

Microemulsion

| | |
|---|---|
| diuron | 0.9% |
| mesotrione | 0.1% |
| triacetine | 30.0% |
| C₈-C₁₀ alkylpolyglycoside | 30.0% |
| glyceryl monooleate | 19.0% |
| water | 20.0%. |

### Example I

Extruded Granule (Pellet)

| | |
|---|---|
| diuron | 24.0% |
| mesotrione, sodium salt | 1.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0%. |

### Example J

Samples of the Diuron Extruded Granule, Mesotrione Extruded Granule, and Hexazinone Extruded Granule compositions containing the ingredients described below are separately prepared by extrusion of milled premixes moistened with water through a circular die to give after drying extrudates with diameters of 1.0 mm±0.1 mm. The extrudates are sized by tumbling in a rotary sifter to provide granules having lengths of at least 1.2 mm and not greater than 7.2 mm and a mean length of 2.5 mm.

Diuron Extruded Granule

| | |
|---|---|
| diuron | 30.0% |
| anhydrous sodium sulfate | 5.0% |
| crude calcium ligninsulfonate | 5.0% |
| sodium alkylnaphthalenesulfonate | 1.0% |
| calcium/magnesium bentonite | 59.0%. |

Mesotrione Extruded Granule

| | |
|---|---|
| mesotrione | 5.0% |
| anhydrous sodium sulfate | 30.0% |
| crude calcium ligninsulfonate | 5.0% |
| sodium alkylnaphthalenesulfonate | 1.0% |
| calcium/magnesium bentonite | 59.0%. |

Hexazinone Extruded Granule

| | |
|---|---|
| hexazinone | 15.0% |
| anhydrous sodium sulfate | 20.0% |
| crude calcium ligninsulfonate | 5.0% |
| sodium alkylnaphthalenesulfonate | 1.0% |
| calcium/magnesium bentonite | 59.0%. |

Then 250 g of the Diuron Extruded Granule composition, 80 g of the Mesotrione Extruded Granule composition and 140 g of the Hexazinone Extruded Granule composition are combined and thoroughly mixed by being placed in a bottle which is inverted until visually homogeneous and thereafter shaken on a mechanical shaker for 15 minutes to provide a homogeneous mixture with a composition of 16.0% diuron, 0.8% mesotrione and 4.5% hexazinone. Alternatively, 250 g of the Diuron Extruded Granule composition and 80 g of the Mesotrione Extruded Granule composition are combined and thoroughly mixed by being placed in a bottle which is inverted until visually homogeneous and thereafter shaken on a mechanical shaker for 15 minutes to provide a homogeneous mixture with a composition of 22.7% diuron and 1.2% mesotrione.

Mixtures and compositions comprising diuron and mesotrione can be used without additional biologically active compounds or agents or can be combined with one or more additional biologically active compounds or agents such as other herbicides, herbicide safeners, insecticides, insecticide synergists, fungicides, nematocides, bactericides, acaricides, growth regulators such as rooting stimulants, chemosterilants, semiochemicals, repellents, attractants, pheromones, feeding stimulants, other biologically active compounds including plant nutrients (i.e. fertilizers) or bacteria, virus or fungi pathogenic to plant, arthropod, nematode, bacterial or fungal pests to form a multi-component pesticide giving an even broader spectrum of agricultural utility. Combination of mixtures of diuron and mesotrione with other biologically active compounds or agents typically preserves the synergism between diuron and mesotrione for controlling weeds. Combination of mixtures of diuron and mesotrione with other herbicides can broaden the spectrum of activity against additional weed species, and suppress the proliferation of any resistant biotypes, and may enhance the synergistic (i.e. greater-than-additive) effect on weeds and/or provide a safening (i.e. less-than-additive) effect on crops or other desirable plants. Combination of one or more of the following herbicides in the mixture and composition of this invention may be particularly useful for weed control: acetochlor, acifluorfen and its sodium salt, aclonifen, acrolein (2-propenal), alachlor, alloxydim, ametryn, amicarbazone, amidosulfuron, aminopyralid, amitrole, ammonium sulfamate, anilofos, asulam, atrazine, AVH-301 (2-[2-chloro-4-(methylsulfonyl)-3-[[(tetrahydro-2-furanyl)methoxy]methyl]benzoyl]-1,3-cyclohexanedione), azimsulfuron, beflubutamid, benazolin, benazolin-ethyl, bencarbazone, benfluralin, benfuresate, bensulfuron-methyl, bensulide, bentazone, benzobicyclon, benzofenap, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clethodim, clodinafop-propargyl, clomazone, clomeprop, clopyralid, clopyralid-olamine, cloransulam-methyl, CUH-35 (2-methoxyethyl 2-[[[4-chloro-2-fluoro-5-[(1-methyl-2-propynyl)oxy]phenyl](3-fluoro-benzoyl)amino]carbonyl]-1-cyclohexene-1-carboxylate), cumyluron, cyanazine, cycloate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, DNOC, endothal, EPTC, esprocarb, ethalfluralin, ethametsulfuron-methyl, ethofumesate, ethoxyfen, ethoxysulfuron, etobenzanid, fenoxaprop-ethyl, fenoxaprop-P-ethyl, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, flucarbazone, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, fluorochloridone, fluoroxypyr, flurtamone, fluthiacet-methyl, fomesafen, foramsulfuron, fosamine-ammonium, glufosinate, glufosinate-ammonium, glyphosate and its salts such as ammonium, isopropylammonium, potassium, sodium (including sesquisodium) and trimesium (alternatively named sulfosate), halosulfuron-methyl, haloxyfop-etotyl, haloxyfop-methyl, hexazinone, HOK-201 (N-(2,4-difluorophenyl)-1,5-dihydro-N-(1-methylethyl)-5-oxo-1-[(tetrahydro-2H-pyran-2-yl)methyl]-4H-1,2,4-triazole-4-carboxamide), imazamethabenz-methyl, imazamox, imazapic, imazapyr, imazaquin, imazaquin-ammonium, imazethapyr, imazethapyr-ammonium, imazosulfuron, indanofan, iodosulfuron-methyl, ioxynil, ioxynil octanoate, ioxynil-sodium, isoproturon, isouron, isoxaben, isoxaflutole, isoxachlortole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, metam-sodium, metamifop, metamitron, metazachlor, methabenzthiazuron, methylarsonic acid and its calcium, monoammonium, monosodium and disodium salts, methyldymron, metobenzuron, metobromuron, metolachlor, S-metholachlor, metosulam, metoxuron, metribuzin, metsulfuron-methyl, molinate, monolinuron, naproanilide, napropamide, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxyamid, phenmedipham, picloram, picloram-potassium, picolinafen, pinoxaden, piperophos, pretilachlor, primisulfuron-methyl, prodiamine, profoxydim, prometon, prometryn, propachlor, propanil, propaquizafop, propazine, propham, propisochlor, propoxycarbazone, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimsulfuron, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tembotrione, tepraloxydim, terbacil, terbumeton, terbuthylazine, terbutryn, thenylchlor, thiazopyr, thiencarbazone, thifensulfuron-methyl, thiobencarb, tiocarbazil, topramezone, tralkoxydim, tri-allate, triasulfuron, triaziflam, tribenuron-methyl, triclopyr, triclopyr-butotyl, triclopyr-triethylammonium, tridiphane, trietazine, trifloxysulfuron, trifluralin, triflusulfuron-methyl, tritosulfuron and vernolate. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butyl.) Butyl. and Puccinia thlaspeos Schub.

Preferred for a wider spectrum of weeds controlled as well as maintaining the synergism between diuron and mesotrione is a mixture and composition of the present invention containing a three-way combination of diuron, mesotrione and hexazinone.

Mixtures and compositions of this invention can also include herbicide safeners such as benoxacor, BCS (1-bromo-4-[(chloromethyl)sulfonyl]benzene), cloquintocet-mexyl, cyometrinil, dichlormid, 2-(dichloromethyl)-2-methyl-1,3-dioxolane (MG 191), fenchlorazole-ethyl, fenclorim, flurazole, fluxofenim, furilazole, isoxadifen-ethyl, mefenpyr-diethyl, methoxyphenone ((4-methoxy-3-methylphenyl)(3-methylphenyl)methanone), naphthalic anhydride (1,8-naphthalic anhydride) and oxabetrinil to increase safety to certain crops. Antidotally effective amounts of the herbicide safeners can be applied at the same time as the compounds of this invention, or applied as seed treatments. Therefore an aspect of the present invention relates to a herbicidal mixture or composition comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a mixture or composition of this invention wherein seed from which the crop is grown is treated with an antidotally effective amount of a safener. Antidotally effective amounts of safeners can be easily determined by one skilled in the art through simple experimentation.

Mixtures and compositions of this invention can also include plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

Mixtures of diuron and mesotrione are highly active herbicides, providing unexpected utility for control of weeds because of synergy on the weeds. Mixtures of diuron and mesotrione are useful for both preemergent and postemergent weed control. Synergism is obtained from both preemergent and postemergent applications, but the synergistic effect is particularly significant in postemergent treatment, especially on larger weeds, for which diuron alone is less effective than in preemergent treatment of germinating weeds. The synergistic effect is also particularly significant on weeds for which mesotrione and diuron individually do not provide high levels of control at desired application rates and weed growth stage. Such problematic weeds include monocot species such as grasses (Family Poaceae), for example Digitaria species such as Digitaria nuda Schumacher (naked crabgrass) and Digitaria horizontalis Willd. (Jamaican crabgrass), and sedges (Family Cyperaceae), for example Cyperus species such as Cyperus esculentus L. (yellow nutsedge) and Cyperus rotundus L. (purple nutsedge). The present mixtures and compositions can also be used to synergistically control weed biotypes that are resistant to triazine and other herbicides that inhibit photosystem II (PSII).

The herbicidal mixtures and compositions of the present invention can be applied to a wide variety of important agronomic crops, particularly in directed applications to minimize contact with the crop plants. Such important agronomic crops include, but are not limited to, cotton, cranberry, wheat, corn (maize), vegetables such as sweet corn, perennial plantation crops including coffee, cocoa, oil palm, rubber, sugarcane, grapes, fruit trees such as citrus, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Coffee includes both Coffea arabica L. and Coffea canephora Pierre ex Froehner (also known as Coffea robusta L. Linden). Citrus includes, for example, orange (Citrus sinensis (L.) Osbeck), tangerine (Citrus reticulata Blanco), lemon (Citrus limon (L.) Burm. f.), lime (Citrus aurantifolia (Christm.) Swingle), pummelo (Citrus maxima (Burm. f.) Merr.) and grapefruit (Citrus maxima x sinensis). Mixtures of this invention can be used in crops genetically transformed or bred to incorporate resistance to herbicides, express proteins toxic to invertebrate pests (such as Bacillus thuringiensis toxin), and/or express other useful traits. Established crops may be sufficiently tolerant to the present herbicidal mixtures and compositions to allow foliage contact without causing excessive injury. Of note is the method of the present invention wherein the locus of the undesired vegetation is a crop selected from citrus, coffee, maize (Zea mays L.), sugarcane (Saccharum officinarum L.), and cranberry (Vaccinium macrocarpon Ait.). The herbicidal mixtures and compositions of the present invention also have utility for broad-spectrum pre- and/or postemergence weed control in areas where complete control of all vegetation is desired such as around fuel storage tanks, industrial storage areas, farm buildings, parking lots, drive-in theaters, air fields, river banks, irrigation and other waterways, around billboards and highway and railroad structures. Furthermore the herbicidal mixtures and compositions can be used to control weeds after harvest or removal of a crop and in fallow situations.

The present mixture and composition comprising diuron and mesotrione, particularly in further combination with hexazinone, are especially useful for selective control of weeds in sugarcane crops. Mixtures of diuron and hexazinone have long been used for weed control in sugarcane. Although such mixtures of diuron and hexazinone provide excellent control of a broad spectrum of weed species, they are weak on the particular crabgrass species Digitaria nuda. Furthermore other herbicides commercially used for weed control in sugarcane crops are also weak on Digitaria nuda. As result of lack of effective herbicide control and release from competition of other weeds, Digitaria nuda has proliferated and become a serious pest. Purple nutsedge (Cyperus rotundus) also is not well controlled by diuron and hexazinone, and is a serious pest in sugarcane as well as other crops. Although mesotrione alone is also weak on these weed species, the combination of diuron with mesotrione, particularly in combination with hexazinone, has now been discovered to provide control through a strong synergistic effect.

As the mixtures have both preemergent and postemergent herbicidal activity, to control undesired vegetation by killing or injuring the vegetation or reducing its growth, the compounds can be usefully applied by a variety of methods involving contacting a herbicidally effective amount of a mixture of the invention, or a composition comprising said mixture and at least one additional component selected from surfactants, solid diluents and liquid diluents, to the locus of the undesired vegetation, which may include the foliage or other part of the undesired vegetation or the environment of the undesired vegetation such as the soil or water in which the undesired vegetation is growing or which surrounds the seed or other propagule of the undesired vegetation.

The mixture and composition according to the present invention comprises herbicidally effective amounts of diuron and mesotrione, and more particularly, synergistically effective amounts of diuron and mesotrione. Any additional biologically active compound or agent in the mixture or composition is also present in a biologically effective amount. The herbicidal composition of the present invention comprises diuron and mesotrione. The method of controlling undesired vegetation comprises applying a herbicidally effective amount of the mixture or composition to the locus of the undesired vegetation. The herbicidally effective amount of the mixture or composition of this invention is determined by a number of factors. These factors include: formulation selected, method of application, amount and type of vegetation present, growing conditions, etc. Herbicidally effective amounts of mixtures and compositions comprising diuron and mesotrione as well as biologically effective amounts of additional biologically active compounds or agents included in the mixture or composition can be easily determined by one skilled in the art through simple experimentation for the desired level of weed control. Furthermore typical agronomically useful application rates of biologically active compounds or agents are published on product labels and references such as The Pesticide Manual, Thirteenth Edition, C. D. S. Tomlin Ed., BCPC, Hampshire, UK, 2003 and The BioPesticide Manual, Second Edition, L. G. Copping, Ed., British Crop Protection Council, Farnham, Surrey, UK, 2001.

The synergism between diuron and mesotrione is exhibited over a wide range of weight ratios of diuron and mesotrione. Indeed synergism is even evident with amounts of diuron and hexazinone which separately provide relatively little herbicidal effect. Synergistically effective amounts of diuron and mesotrione in the present mixture and composition can be easily determined by one skilled in the art through simple experimentation.

In the mixture and composition of the present invention, diuron and mesotrione are typically present in a weight ratio not exceeding about 400:1 and not less than about 2:1. More typically the weight ratio of diuron to mesotrione is from about 100:1 to about 5:1. Most typically the weight ratio of diuron to mesotrione is from about 30:1 to about 5:1. If hexazinone is present in the mixture or composition, the weight ratio of diuron to hexazinone is typically from about 20:1 to about 2:1 and more typically from about 10:1 to about 3:1. The total weight of diuron and mesotrione in the mixture and composition of the present invention applied to the locus of the undesired vegetation is typically not less than about 0.4 kg/ha or greater than about 10 kg/ha. More typically the total weight of diuron and mesotrione applied is between about 0.8 kg/ha and about 5 kg/ha. Most typically the total weight of diuron and mesotrione applied is between about 0.9 kg/ha and about 4 kg/ha. If hexazinone is present in the mixture or composition, it typically is applied at an application rate between about 0.05 kg/ha and about 3 kg/ha, more typically at an application rate between about 0.1 kg/ha and about 2 kg/ha, and most typically at an application rate between about 0.1 kg/ha and 1 kg/ha.

The following Test demonstrates the control efficacy of the mixtures of this invention against a specific weed. The weed control afforded by the mixtures is not limited, however, to this species.

### Biological Examples of the Invention

### Test A

Square plastic pots (10 cm on each side) were filled to the rim with a Redi-earth® (Sun Gro Horticulture, Bellevue, Wash.) planting medium containing 55-65% of Canadian sphagnum peat moss, horticultural grade vermiculite, dolomitic limestone and a wetting agent. The soil was tamped firm to a uniform level of 2.5 cm below the rim of the pot. A measured amount of Digitaria nuda seed was scattered on the soil surface of each pot. Seeded pots were covered with approximately 50 mL of the same planting media, tamped, and set out on a greenhouse bench.

The pots were watered thoroughly at once and then as needed with a dilute balanced N—P—K fertilizer solution to provide 218 ppm nitrogen. Artificial lighting was used to supplement natural light to produce a 14-hour photoperiod. Greenhouse temperatures were targeted for 27° C. during the daytime and 21° C. at night. Once the plants reached their appropriate growth stage, the plants were sorted into test units prior to the herbicide application. Digitaria nuda was 5 to 7 cm in height and at the 3-leaf stage at the time of application.

Test chemicals included diuron in the form of KARMEX® Herbicide, which is a 80 weight (wt.) % wettable granule formulation, a mixture of diuron with hexazinone in the form of DUPONT™ K4™ Herbicide, which a granule formulation containing 468 g of diuron and 132 g of hexazinone per kg of formulation, another mixture of diuron with hexazinone in the form of ADVANCE™ Herbicide, which is a wettable granule formulation containing 533 g of diuron and 67 g of hexazinone per kg of formulation, and mesotrione in the form of the commercial product CALLISTO® Herbicide, which is a 40 weight-volume (wt.-vol.) % suspension concentrate containing 400 g of mesotrione per liter of formulation. The application rates for this test were selected based on the recommended use rate of each compound. Application rates of diuron were 1800 g a.i./ha alone from KARMEX® Herbicide, 936 g a.i./ha from DUPONT™ K4™ Herbicide in combination with 264 g a.i./ha of hexazinone, and 1599 g a.i./ha from ADVANCE™ Herbicide in combination with 201 g a.i./ha of hexazinone. Application rates of mesotrione were 5, 10, 20, 40 and 60 g a.i./ha from CALLISTO®. Furthermore diuron was tested alone and together with hexazinone in combination with mesotrione at these application rates.

The herbicides were diluted in a measured amount of carrier solution to make stock solutions. The carrier solution was water containing 0.25% v/v of non-ionic adjuvant X-77. The stock solutions were all mixed for 10 minutes before aliquots were taken to make individual treatment solutions. Vials were then brought up to final spray volume of 24 mL. The treatments were sprayed at a pressure of 276 kPa through a T-Jet 8002E (Spraying Systems Co., Wheaton, Ill.) flat fan nozzle onto the surface of the plants in a stationary belt sprayer system calibrated to deliver 280 L/Ha. Treatments were replicated three times.

The sprayed plants were placed in the greenhouse for observation; height and leaf stages were then recorded. Plants were maintained by watering as needed and in the same manner in which they were propagated with respect to nutrition, light, and day length. Visual evaluations were made 21 days after treatment (DAT). A numerical rating system was used to describe the visual response of each treatment on a scale of 0 to 100. Numerical ratings were expressed as the percent effect as compared to the control plants, calculated as the mean of the three replicates. Zero (0%) indicates no visual response, and 100% indicates plant death or a moribund state expected to rapidly end in death.

Colby's equation was used to calculate the expected additive herbicidal effect of the mixtures of mesotrione with diuron, alone and in combination with hexazinone. Colby's equation (S. R. Colby, “Calculating Synergistic and Antagonistic Responses of Herbicide Combinations,” Weeds, 15(1), pp 20-22 (1967)) calculates the expected additive effect of herbicidal mixtures, and for two active ingredients is of the form:

P_{a+b}=Pₐ+P_{b}−(PₐP_{b}/100)

wherein P_{a+b} is the percentage effect of the mixture expected from additive
contribution of the individual components,
Pₐ is the observed percentage effect of the first active ingredient at the same use rate as in the mixture, and
P_{b} is the observed percentage effect of the second active ingredient at the same use rate as in the mixture.
   For mixtures comprising diuron, hexazinone and mesotrione, the expected herbicidal effect was calculated based on the combination of diuron with hexazinone providing observed herbicidal effect Pₐ and mesotrione providing observed herbicidal effect P_{b} in the Colby Equation.

Observed and expected herbicidal effects are listed in Table A.

TABLE A (*)
Effect of Diuron, Hexazinone Combinations and Mesotrione
Alone and in Mixtures on Digitaria nuda

| Application Rate (g a.i./ha) | | | Herbicidal Effect | |
|---|---|---|---|---|
| Diuron | Hexazinone | Mesotrione | Observed | Expected |
| 1800 | 0 | 0 | 20 | — |
| 936 | 264 | 0 | 13 | — |
| 1599 | 201 | 0 | 10 | — |
| 0 | 0 | 5 | 3 | — |
| 0 | 0 | 10 | 17 | — |
| 0 | 0 | 20 | 33 | — |
| 0 | 0 | 30 | 50 | — |
| 0 | 0 | 40 | 53 | — |
| 0 | 0 | 60 | 73 | — |
| 0 | 0 | 90 | 88 | — |
| 1800 | 0 | 5 | 30 | 22 |
| 1800 | 0 | 10 | 43 | 33 |
| 1800 | 0 | 20 | 78 | 47 |
| 1800 | 0 | 30 | 83 | 60 |
| 1800 | 0 | 40 | 95 | 63 |
| 1800 | 0 | 60 | 100 | 79 |
| 936 | 264 | 5 | 30 | 16 |
| 936 | 264 | 10 | 43 | 28 |
| 936 | 264 | 20 | 73 | 42 |
| 936 | 264 | 30 | 95 | 57 |
| 936 | 264 | 40 | 100 | 60 |
| 936 | 264 | 60 | 100 | 77 |
| 1599 | 201 | 5 | 16 | 13 |
| 1599 | 201 | 10 | 30 | 25 |
| 1599 | 201 | 20 | 50 | 40 |
| 1599 | 201 | 30 | 68 | 55 |
| 1599 | 201 | 40 | 100 | 58 |
| 1599 | 201 | 60 | 100 | 76 |

(*) Application rates are expressed as grams of active ingredient per hectare (g a.i./ha) for diuron, hexazinone and mesotrione. Observed herbicidal effect is reported as percent control based on mean responses from three replicates. Expected herbicidal effect is calculated according to the method of S. R. Colby, “Calculating Synergistic and Antagonistic Responses of Herbicide Combinations,” Weeds, 15(1), pp 20-22 (1967).

As can be seen from the observed and expected herbicide test results in Table A, mixtures of diuron and mesotrione provided a greater herbicide effect than expected from their individual effects. The synergistic effect occurred over a range of diuron application rates (936, 1599 and 1800 g a.i./ha) and mesotrione application rates (5, 10, 20, 30, 40 and 60 g a.i./ha). This synergistic effect occurred irrespective of whether diuron was in combination with hexazinone. Indeed the combinations including hexazinone in addition to diuron appeared to demonstrate an even stronger synergism with mesotrione, thus indicating that addition of hexazinone to the diuron and mesotrione not only maintained the synergism but further increased it.

What is claimed is:

1. A mixture comprising diuron and at least one compound selected from mesotrione and salts thereof.

2. The mixture of claim 1 further comprising at least one additional biologically active compound or agent.

3. The mixture of claim 2 wherein said at least one additional biologically active compound or agent comprises hexazinone.

4. A herbicidal composition comprising diuron, at least one compound selected from mesotrione and salts thereof, and at least one additional component selected from the group consisting of surfactants, solid diluents and liquid diluents.

5. The herbicidal composition of claim 4 further comprising at least one additional biologically active compound or agent.

6. The herbicidal composition of claim 5 wherein said at least one additional biologically active compound or agent comprises hexazinone.

7. The herbicidal composition of claim 4 which is a homogeneous mixture comprising two groups of substantially cylindrical granules, the groups having different herbicide contents, the first group of granules comprising diuron and at least one solid diluent, and the second group of granules comprising at least one compound selected from mesotrione and salts thereof and at least one solid diluent, the granules within each group having substantially uniform diameters and longitudinal lengths of from 1 to 8 times the diameter with the average length of the granules being from 1.5 to 4 times the diameter, and the average diameter of the second group differing from the first group by no more than 30%.

8. The herbicidal composition of claim 7 wherein the homogeneous mixture further comprises a third group of substantially cylindrical granules having a herbicide content differing from the herbicide contents of the first and second groups, the third group of granules comprising hexazinone and at least one solid diluent, the granules within the third group having substantially uniform diameters and longitudinal lengths of from 1 to 8 times the diameter with the average length of the granules being from 1.5 to 4 times the diameter, and the average diameter of the third group differing from the first and second groups by no more than 30%.

9. A method of controlling undesired vegetation comprising applying to the locus of the vegetation a herbicidally effective amount of the mixture of any one of claims 1, 2 or 3.

10. The method of claim 9 wherein the locus of the vegetation is a citrus, coffee, maize, sugarcane or cranberry crop.

11. The method of claim 10 wherein the locus of the vegetation is a sugarcane crop.

12. The method of claim 9 wherein the locus of the vegetation is other than a crop.
